SEQUENCE LISTING (4 sequences)

SEQ ID NO: 1 - DNA, 562 bp, Homo sapiens
  CDS (1)..(432)
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgcccc

SEQ ID NO: 2 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 3 - DNA, 753 bp, Mus musculus
  CDS (39)..(461)
aaaccgaactccaggaccagagacccagagatcagaggatgcagcaccgaggcttcttccttctcgcccttcttgccctcttggtggtcacgtccgcggtggccaaaaaaaaagagaaggtgaagaagggcagcgagtgttcggagtggacctgggggccctgcacccccagcagcaaggactgcggcatgggcttccgcgagggtacctgtggggcccagacccagcgcgtccattgcaaggtgccctgcaactggaagaaggaatttggagccgactgcaaatacaagtttgagagctggggggcgtgtgatgggagcactggcaccaaagcccgccaagggaccctgaagaaggcgcggtacaatgcccagtgccaggagaccatccgcgtgactaagccctgcacctccaagaccaagtcaaagaccaaagccaagaaaggaaaaggaaaggactaagtcaggaggccagagagcctccggcctcgcctggagcctgaacggagccctcctctcccacaggcccaagatataacccaccagtgccttttgtcttcctgtcagctctgtcaatcacgcctgtcctctcacgcccacaccaagtgcccaaagtggggagggacaagagattctggaaagtgagcctccccataccctcttttgttctccccaccctgatacttgctattaagaaatgaataaaataaactcacttttttccaataaaaaaaaaaaaaaaaaaaaaaaaaaa

SEQ ID NO: 4 - protein, 140 aa, Mus musculus
MQHRGFFLLALLALLVVTSAVAKKKEKVKKGSECSEWTWGPCTPSSKDCGMGFREGTCGAQTQRVHCKVPCNWKKEFGADCKYKFESWGACDGSTGTKARQGTLKKARYNAQCQETIRVTKPCTSKTKSKTKAKKGKGKD

(19) United States
(12) Patent Application Publication
(10) Pub. No.: US 2010/0105613 A1
(43) Pub. Date: Apr. 29, 2010

(54) THERAPEUTIC AGENT FOR OCCLUSIVE PERIPHERAL VASCULAR DISEASE, AND USE THEREOF

(76) Inventors: Mitsuru Horiba, Nagoya-Shi (JP); Itsuo Kodama, Nagoya-Shi (JP); Kenji Kadomatsu, Nagoya-Shi (JP)

Correspondence Address: MORRISON & FOERSTER LLP, 12531 HIGH BLUFF DRIVE, SUITE 100, SAN DIEGO, CA, 92130-2040, US

(21) Appl. No.: 12/446,446

(22) PCT Filed: Oct. 19, 2007
(86) PCT No.: PCT/JP2007/070441

§ 371 Date: Dec. 17, 2009

(30) Foreign Application Priority Data
Oct. 20, 2006 (JP) 2006-286848

(51) Int. Cl. A61K 38/16; A61K 31/7052; A61P 9/00

(52) U.S. Cl. 514/12; 514/44 R

(57) ABSTRACT

A study was made on the therapeutic effect of midkine on an occlusive peripheral vascular disease, and it was found that midkine has an activity of promoting neovascularization and that a blood vessel can be proliferated and the blood flow in the upper and lower limbs can be improved (in other words, the condition of an ischemic disease in the upper and lower limbs can be ameliorated) by introducing midkine into a site affected by the occlusive peripheral vascular disease.

### FIELD OF THE INVENTION

The present invention is directed to agents for treating or preventing occlusive peripheral vascular diseases, comprising midkine as an active ingredient, and to uses thereof.

### BACKGROUND OF THE INVENTION

Midkine (hereinafter may be referred to as “MK”) which belongs to a heparin-binding growth factor family is a low-molecular-weight nonglycosylated protein found as a product of a retinoic acid-responsive gene. A receptor of MK has been considered to be a complex of a receptor type tyrosine phosphatase z, LRP (low density lipoprotein receptor-related protein), ALK (anaplastic leukemia kinase) and syndecan. MK has been appeared to have activities for cell migration and angiogenesis as well as diverse bioactivities in induction of canceration and inflammation. It has also been reported that MK overexpresses in numerous carcinoma tissues such as gastric cancer, colon cancer and breast cancer (Tsutsui, J. et al., Cancer Res., 53, 1281-1285 (1993) and Kadomatsu, K. et al., Brit. J. Cancer, 75, 354-359 (1997)) . Meanwhile, there have also been reports of disorders in vascular intima and ischemic renal disorder in MK-deficient mice (Horiba, M., et al. J. Clin. Invest., 105, 489-495 (2000) and Sato, W., et al. J. immunol., 167, 3463-3469 (2001)).

Ischemia is a disease state wherein blood flow is completely-interrupted or drastically-decreased at a part of the body, causing simultaneous progression of oxygen deficiency, reduced substrate supply, and accumulation of metabolites. Severity of ischemia depends on the extent or duration of vascular occlusion, sensitivity of the tissue or extent of bypass development. A functional disorder may occur in an organ or tissue caused ischemia, and the prolonged ischemia leads to the atrophy, degeneration or necrosis of the tissue.

In recent years, the number of occurrences of lower extremity ischemic diseases based on arteriosclerosis obliterans (ASO) or Buerger's disease (TAO) underlying arteriosclerosis has increased with progression of westernization of dietary habits or ageing (Dormandy J. A. et al., J. Vasc Surg., 31, S1-S296 (2000)). In Japan, the incidence of chronic limb ischemia is 50-100 per 100,000 people per year, and around 5% of 60-70-year-old men have symptoms of intermittent claudication, in which, although 50-75% can be followed up without therapy, leg amputation is considered to be indicated in 1% per year.

In recent years, development of revascularization procedures, such as medical therapies with drugs (Bendermacher B. L. et al., J. Thromb. Haemost., 3(8), 1628-1637 (2005) and Hankey G. J. et al., JAMA., 295(5), 547-553 (2006)), surgical therapies (Willigendael E. M. et al., J. Vasc. Surg., 42, 67-74 (2005) and Lauterbach S. R. et al., Arch. Surg., 140(5), 487-493 (2005)) and intervention therapies for vascular stenosis sites using a cutting balloon and a rotablator (Cejna M., Cardiovasc. Intervent. Radiol., 28, 400-408 (2005) and Dormal P. A. et al., Acta Chir. Belg., 105(2), 231-234 (2005)) has increased a lifesaving rate of patient suffering from occlusive peripheral vascular disease. In contrast, however, the number of aged patients with severe limb ischemia having complicated lesions has been increased in recent years. Such patients have no choice but to undergo leg amputation, resulting in significantly-impaired quality of life and poor prognosis, and the five-year survival rate of these patients is reported as 50%.

Therapeutic angiogenesis using gene transfer, cell transplantation or growth factor protein introduction has been attempted for patients who cannot be expected to be improved by conventional medical/surgical therapies, however decisive therapeutic effects have not yet been achieved.

Further, information on prior art documents according to the present invention is shown below.

non-patent document 1

### BRIEF SUMMARY OF THE INVENTION

The present invention was invented with respect to such circumstances and is directed to provide an agent for treating or preventing occlusive peripheral vascular diseases comprising midkine as an active ingredient. The present invention is also directed to provide a method of treating or preventing occlusive peripheral vascular diseases, comprising a step of administering midkine to a subject.

The inventors examined the therapeutic effects of administration of midkine on occlusive peripheral vascular diseases in order to solve the aforementioned problems.

First, it was examined whether or not growth factor midkine showed angiogenic activity. Specifically, angiogenic activities were compared in subcutaneous matrigel injection of midkine into mice. As a result, the remarkable proliferation of the blood vessels by the addition of the midkine was observed in comparison with the controls (FIG. 1). In addition, the number of blood vessels measured at 7 days after the administration exhibited a significant increase compared to the controls receiving no substance (FIG. 2).

Subsequently, it was examined whether or not midkine had a lumen formation activity in cultured vascular endothelial cells. Midkine was added to HUVECs (normal human umbilical vein endothelial cells) cultured on growth factor-deficient matrigel, and its influence on lumen formation was observed. As a result, the lumen formation was clearly promoted by the midkine, as in the case of bFGF used as the positive control (FIG. 3). These results have strongly supported that midkine has angiogenic activity.

Subsequently, the therapeutic effect of midkine was examined in occlusive peripheral vascular disease model mice. Specifically, lower extremity ischemic disease model mice were produced, and the blood flow at the lower extremity was measured in each of the midkine-knockout and wild-type mice. As a result, on postoperative day 14, the blood flows of the midkine-knockout mice were found to significantly decrease compared to the wild-type mice (FIG. 5). This result revealed that midkine is involved in peripheral revascularization.

Furthermore, adenovirus vectors were used to introduce midkine into the ischemic lower extremity muscles of the lower extremity ischemic disease model wild-type mice. As a result, significant improvement in blood flow was observed in the group which midkine was introduced compared to the non-treatment group (group receiving an injection of only adenovirus vectors) (FIG. 6).

In addition, midkine was introduced into the ischemic lower extremities of rats as a sustained-release preparation using fine hydroxyapatite particles or gelatine in order to verify the therapeutic effects of midkine on lower extremity ischemic diseases. In such rats, angiogenesis and significant improvement in blood flow were observed and prevention of the necrosis of the lower extremity was found (FIGS. 7-10).

Furthermore, the morphologic evaluation of the angiogenesis due to the midkine revealed that slight malformation (vascular malformation) of the microvessels was generated by the midkine compared to the continuous VEGF treatment group (FIG. 11).

In other words, the present inventors achieved the present invention by finding that midkine has the activity of promoting angiogenesis and that introduction of midkine into affected area of a lower extremity ischemic disease permits proliferation of the blood vessels to improve the blood flow of the lower extremity and to improve the symptom of the lower extremity ischemic disease.

More specifically, the present invention provides the following [1] to [9]:

[1] an agent for treating or preventing an occlusive peripheral vascular disease, comprising midkine or its analog as an active ingredient;
[2] the agent according to [1], wherein the agent is a sustained-release preparation with fine hydroxyapatite particles or gelatine;
[3] an agent for treating or preventing an occlusive peripheral vascular disease, comprising a virus vector comprising at least one DNA that encodes midkine or its analog as an active constituent;
[4] the agent according to any one of [1] to [3], wherein the occlusive peripheral vascular disease is based on arteriosclerosis obliterans, Buerger's disease or diabetic angiopathy;
[5] a method of treating or preventing an occlusive peripheral vascular disease, comprising a step of administering midkine or its analog to a subject;
[6] the method according to [5], wherein the midkine or its analog is administered as a sustained-release preparation with fine hydroxyapatite particles or gelatine;
[7] the method according to [5] or [6], wherein the midkine or its analog is administered by intravenous or intramuscular injection;
[8] a method of treating or preventing an occlusive peripheral vascular disease, comprising a step of administering a virus vector comprising at least one DNA that encodes midkine or its analog; and
[9] the method according to any one of [5] to [8], wherein the occlusive peripheral vascular disease is based on arteriosclerosis obliterans, Buerger's disease or diabetic angiopathy.

### BRIEF DESCRIPTION OF DRAWINGS

FIG. 1 shows a photograph showing the angiogenic activity of midkine in subcutaneous matrigel.

FIG. 2 shows photographs and a graph, of the results of the number of blood vessels in subcutaneous matrigel.

FIG. 3 shows photographs exhibiting the lumen formation activity of midkine for cultured vascular endothelial cells.

FIG. 4 shows a figure exhibiting a site of removal of the blood vessels in a lower extremity ischemic disease model mouse.

FIG. 5 shows a graph of the comparison of the blood flows of the lower extremities in two groups of lower extremity ischemic disease model mouse (midkine-knockout mouse group and wild-type mouse group).

FIG. 6 shows photographs and a graph which show exhibiting the comparison of the blood flow of the lower extremities in two groups of lower extremity ischemic disease model mice (midkine gene-bearing adenovirus vector treatment group and non-treatment group).

FIG. 7 shows photographs exhibiting therapeutic effects of MK administration on the ischemic lower extremities. Necrosis and loss of the ischemic lower extremities, which were often seen in the control, were suppressed in the MK treatment (with gelatine) group.

FIG. 8 shows a graph exhibiting the survival period and number of the ischemic lower extremities in the MK treatment and control groups. The survival period and number of the ischemic lower extremities were improved by the MK treatment (using gelatine).

FIG. 9 shows photographs and a graph of the results of the therapeutic effect of MK by a blood flowmeter. The improvement of the blood flow by the MK treatment (with gelatine) was observed.

FIG. 10 shows photographs and a graph showing immunostaining of the adductor muscle at the ischemic lower extremities area of rats with von Willebrand factor. An increase in the number of new blood vessels by MK treatment (with Hap) can be seen.

FIG. 11 shows photographs and a graph exhibiting the results of continuous administration of MK and VEGF proteins to the auricles of mice. An increase in the number of capillary vessels is observed in the MK treatment group, compared to the control. In the VEGF treatment group, although an increase in the number of blood vessels is also observed, a lot of vascular anomalies were seen in comparison with the MK group.

### DETAILED DESCRIPTION OF THE INVENTION

The present inventors found that midkine has the activity of promoting angiogenesis and that administration of midkine to a lesion with an occlusive peripheral vascular diseases permits proliferation of the blood vessels to improve the blood flow of the peripheral vessel and to improve the symptom of the occlusive peripheral vascular diseases. The present invention is based on these findings.

The present invention relates to agents for treating or preventing occlusive peripheral vascular diseases, comprising midkine or its analog as an active ingredient.

For the present invention, “midkine or its analog” refers to midkine or a protein functionally equivalent to midkine. Midkines may include, for example, human-derived midkine including a protein which is encoded by a cDNA sequence shown in SEQ ID NO: 1 or has an amino acid sequence shown in SEQ ID NO: 2, and mouse-derived midkines including a protein which is encoded by a cDNA sequence shown in SEQ ID NO: 3 or has an amino acid sequence shown in SEQ ID NO: 4.

Proteins functionally equivalent to midkine include, for example, but are not limited to the variants, homologs or partial peptides of midkine, or proteins fused to other proteins. Midkines for the present invention can also include proteins belonging to “midkine family” having all such modifications and alterations, (such as pleiotrophin), as long as the proteins possess the original bioactivity.

The aforementioned protein functionally equivalent to midkine usually comprises an amino acid sequence with high homology to the midkine. The high homology refers to typically at least 50% identity, preferably at least 75% identity, more preferably at least 85% identity, further preferably at least 95% identity in amino acid sequence. An identity in amino acid sequence or base sequence may be determined by BLAST (Proc. Natl. Acad. Sci. USA 90:5873-5877, 1993), or the like.

In the present invention, the number of mutated amino acids is not limited but it may be typically within 30 amino acids, preferably within 15 amino acids, more preferably within 5 amino acids (for example, within 3 amino acids). Amino acid residues are desirably mutated to other amino acids having side chains retaining the properties of the original amino acid side chains. Properties of amino acid side chains may include, for example, hydrophobic amino acids, hydrophilic amino acids, amino acids having an aliphatic side chain, amino acids having a side chain containing a hydroxy group, amino acids having a side chain containing a sulfur atom, amino acids having a side chain containing carboxylic acid and an amide, amino acids having a side chain containing a base, and amino acids having a side chain containing an aromatic group. A polypeptide having an amino acid sequence modified by deletion of one or more amino acid residues from a specific amino acid sequence, addition of one or more amino acid residues to a specific amino acid sequence, and/or substitution by another amino acid has been already known to maintain its biological activity.

In accordance with the present invention, “functionally equivalent” means that a protein of interest has a biological or biochemical function equivalent to or greater than that of midkine. In the present invention, the biological and biochemical functions of midkine include promotion of cell proliferation (promotion of fibroblast, keratinocyte or tumor cell proliferation), enhancement of cell survival (enhancement of survival of fetal neurons or tumor cells), promotion of cell migration (promotion of migration of neurons, neutrophils, macrophages, osteoblasts or vascular smooth muscle cells), promotion of chemokine expression, promotion of angiogenesis, promotion of synaptogenesis, or the like. The biological and biochemical function of midkine in the present invention is preferably promotion of angiogenesis. Biological properties include, e.g., specificity of expression site and expression level.

Methods of obtaining “a protein functionally equivalent to midkine” well-known to those skilled in the art include, for example, a method comprising obtaining a protein coded by a similar base sequence from a naturally-occurring or artificially-altered protein using the hybridization technique or the polymerase chain reaction (PCR) technique. A protein functionally equivalent to midkine can be also obtained by artificial introduction of a mutation using midkine as a lead protein. DNA encoding a protein functionally equivalent to midkine isolated by the above mentioned technique is also included in DNA that encodes midkine or its analog of the present invention.

A hybridization reaction is employed, preferably under stringent conditions, to isolate such DNA. In the present invention, the stringent hybridization conditions refer to hybridization conditions of 6 M urea, 0.4% SDS and 0.5×SSC, or an equivalent stringency hybridization conditions thereto. Even more highly homologous DNA can be expected to be isolated by employing even more highly stringent conditions, e.g., 6 M urea, 0.4% SDS and 0.1×SSC. DNA isolated in such a manner is believed to be highly homologous at the amino acid level to the amino acid sequence of a protein of interest. The expression “highly homologous” refers to a sequence identity of at least 50% or higher, preferably 70% or higher, further preferably 90% or higher (e.g., 95%, 96%, 97%, 98%, 99% or higher) in the overall amino acid sequence. The identities of amino acid sequences and base sequences can be determined by using the BLAST algorithm developed by Carlin and Altschul (Proc. Natl. Acad. Sci. USA 87:2264-2268, 1990; Proc. Natl. Acad. Sci. USA 90:5873, 1993) . Programs based on the BLAST algorithm such as BLASTN and BLASTX have been developed (Altschul S F, et al: J Mol Biol 215:403, 1990). Specific techniques for these analytical methods are well-known.

Species from which midkine or its analog of the present invention may be derived are not limited to specific species and include, for example, human, monkey, mouse, rat, guinea pig, swine, bovine, and the like.

Any production system can be used for producing midkine or its analog of the present invention. Production systems for producing midkine include in vitro and in vivo production systems. In vitro production systems include systems employing eucaryotic cells and those employing procaryotic cells.

In using production systems employing eucaryotic cells, animal cells, plant cells or fungal cells can be employed. As such animal cells: (1) mammalian cells, such as CHO, COS, myeloma, BHK (baby hamster kidney), HeLa and Vero cells; or (2) insect cells, such as sf9, sf21 and Tn5 cells are known. As such fungal cells, for example, Pichia pastoris; S. pombe, e.g., Saccharomyces such as Saccharomyces cerevisiae; and filamentous fungi, e.g., Aspergillus such as Aspergillus niger are known.

Production systems employing procaryotic cells include those that employ bacterial cells. As such bacterial cells, E. coli and Bacillus subtilis are known.

Midkine is obtained by introducing a midkine gene of interest into these cells by means of transformation, and culturing the transformed cells in vitro. The cells are cultured in accordance with well-known methods. For example, DMEM, MEM, RPMI 1640 and IMDM may be used as the culture medium optionally in combination with a serum supplemental solution such as fetal calf serum (FCS). Midkine may also be produced in vivo by transferring the cells introduced midkine genes into the abdominal cavity or other part of an animal.

In contrast, in vivo production systems include those that employ animals or plants. Production systems that employ animals include those that employ mammals or insects.

Mammals, such as goat, swine, sheep, mouse and bovine, may be used in vivo production systems (Vicki Glaser, SPECTRUM Biotechnology Applications, 1993) . Also, insects such as silkworms may be used. And plants such as Nicotiana tabacum may be used.

After introducing midkine genes into these animals or plants, midkine proteins are produced in the bodies of the animals or plants, and then collected.

Midkine produced and expressed as described above may be isolated from outside/inside of the cells or the host, and be purified to homogeneity. The isolation and purification of midkine as used in the present invention may be achieved using affinity chromatography. Other isolation and purification methods used for standard protein may also be employed without any limitation.

The concentration of the midkine obtained as described above may be determined by methods such as absorbance measurements or ELISA.

In the present invention, “occlusive peripheral vascular diseases” can include arteriosclerosis obliterans, Buerger's disease, and diseases based on diabetes mellitus (diseases occurring as complications). Occlusive sites, which are not particularly limited but are peripheral vessels, are preferably peripheral lower extremity vessels.

The agent of the present invention may be used not only for a subject (patient)) with a severe occlusive peripheral vascular disease but also for a subject (patient)) with a developing and mild occlusive peripheral vascular disease.

A pharmaceutically acceptable additives such as preservatives or stabilizers may be added to the agent comprising midkine as an active ingredient of the present invention. The expression “pharmaceutically acceptable” means that the additive itself may possess a therapeutic effect for the aforementioned occlusive peripheral vascular diseases or may not posess the therapeutic effect, and that the additive is a material which is pharmaceutically acceptable and can be administered in combination with the aforementioned therapeutic agents. Also, the additive may possess no therapeutic effect for occlusive peripheral vascular diseases but possess a synergistic or additional stabilization effect by using in combination with midkine.

For example, pharmacologically acceptable additives include sterilized water, physiological saline solution, stabilizers, excipients, buffering agents, preservatives, surfactants, chelating agents (e.g., EDTA) and binders.

Surfactants of the present invention include nonionic surfactants such as sorbitan fatty acid esters, glycerin fatty acid esters, polyglyceryl fatty acid esters and polyoxyethylene sorbitan fatty acid esters.

Surfactants include anionic surfactants such as alkyl sulfates; polyoxyethylene alkyl ether sulfates; alkyl sulfosuccinate ester salts; naturally-occurring surfactants such as lecithin, glycerophospholipid and sphingophospholipid; and sucrose fatty acid esters.

One of these surfactants or a combination of two or more thereof may be added to the agent of the present invention. Preferably, surfactant used in the formulation of the present invention is polyoxyethylene sorbitan fatty acid esters, such as polysorbates 20, 40, 60 or 80. Polyoxyethylene polyoxypropylene glycols as typified by poloxamer such as Pluronic F-68 (Registered Trademark) are also preferable.

Buffering agents used in the present invention include phosphoric acid, citric acid buffer solution and other organic acids; and carbonate buffer solution, Tris buffer solution and the like.

Solution formulations may be prepared by dissolving the agent in an aqueous buffer solution well-known in the field of solution formulation.

The agent of the present invention may contain other ingredients such as polypeptides of low molecular weight, serum albumin, proteins such as gelatine and immunoglobulin, amino acids, sugars and carbohydrates such as polysaccharides and monosaccharides, and sugar alcohols.

In the present invention, amino acids include basic amino acids such as arginine, lysine, histidine and ornithine, and inorganic salts thereof. In case of using free amino acids, a preferred pH value is adjusted by adding appropriate physiologically acceptable buffer additives such as inorganic acid, specifically, hydrochloric acid, phosphoric acid, sulfuric acid, acetic acid or formic acid, or salts thereof. Preferably, amino acids are arginine, lysine, histidine and ornithine. Acidic amino acid, neutral amino acid or aromatic amino acid may be also used.

For example, sugars and carbohydrates such as polysaccharides and monosaccharides used in the present invention include dextran, glucose and fructose.

For example, sugar alcohols used in the present invention include mannitol, sorbitol and inositol.

In employing an injectable aqueous solution form, e.g., physiological saline solution or isotonic solution containing glucose or other additives may be also used concurrently.

The agent may also contain diluents, solubilizing agents, pH adjusters, soothing agents, sulfur-containing reducing agents, antioxidants, and the like, if desired.

In the present invention, sulfur-containing reducing agents include, for example, N-acetylcysteine, N-acetyl homocysteine, thioctic acid, and the like.

In the present invention, antioxidants include, for example, chelating agents such as erythorbic acid, dibutyl hydroxytoluene, butylhydroxyanisol and α-tocopherol.

Furthermore, the agent may be contained in microcapsules (microcapsules formed of hydroxymethyl cellulose, gelatine, poly[methyl methacrylic acid], and the like), or may be delivered in the form of a colloidal drug delivery system (liposome, albumin microsphere, microemulsion, nanoparticles, nanocapsules, and the like) if needed (see “Remington's Pharmaceutical Science 16^{th} Edition”, Oslo Ed., 1980, etc.). Methods for forming the agent into sustained-release formulation are also well-known, and may be applied to the present invention (Langer et al., J. Biomed. Mater. Res. 1981, 15: 167-277; Langer, Chem. Tech. 1982, 12:98-105; U.S. Pat. No. 3,773,919; European Patent Publication Nos. EP 58481 and EP 133988; and Sidman et al., Biopolymers 1983, 22:547-556).

Pharmaceutically acceptable carriers used in the present invention may be selected from the above-mentioned materials depending on the form of a drug, either appropriately or in combination, but are not limited thereto.

The present invention also relates to a method of treating or preventing occlusive peripheral vascular diseases, comprising a step of administering midkine to a subject.

All agents in accordance with the present invention may be administered in the form of pharmaceutical preparations and may be administered either orally or parenterally, and either systemically or locally (directly to lesions with occlusive peripheral vascular diseases). For example, routes of administration of the agent of the present invention include intravenous injection such as intravenous drip, intramuscular injection, subcutaneous injection, suppository, intestinal infusion and oral administration. The route of administration of the agent of the present invention may be selected appropriately depending on the age and symptoms of the patient. The effective dosage may be selected from the range of 0.001 mg to 1000 mg per kg of body weight, preferably 0.005 mg to 100 mg, more preferably 0.01 mg to 50 mg. For example, when the agent is midkine protein, the preferred dosage is such an effective dosage level that free antibodies are found in the blood. Specifically, for example, the preferred dosage and administration is a dose of 0.5 mg to 40 mg, preferably 1 mg to 20 mg per kg of body weight per month (4 weeks), either in a single dosage or divided into several dosages, with an administration schedule, e.g., twice weekly, once weekly, once biweekly, or once every four weeks, by intravenous injection such as intravenous drip, subcutaneous injection or intramuscular injection.

In the present invention, “lesion with occlusive peripheral vascular disease” refers to a site including an ischemic lesion and a peripheral part thereof. To the ischemic lesion, intravascular or intramuscular administration may be carried out, particularly intramuscular administration to the ischemic lesion is preferably carried out. More specifically, in occlusive peripheral vascular diseases, the administration of the agent into the skeletal muscles of the ischemic lesions of the upper or lower extremities permits the promotion of angiogenesis in the ischemic lesions to improve blood flow and the functional recovery and to normalize the ischemic lesions.

The agent of the present invention may be also administered with a well-known factor having angiogenic activity. Well-known factors having angiogenic activity include, but are not limited to, factors such as VEGF, FGF, HGF and EGF.

The agent of the present invention may be also continuously administered to a subject. Methods of continuously administering the agent of the present invention include a method of subcutaneously implanting a sustained-release capsule (sustained-release preparation) of fine hydroxyapatite particles into which midkine protein is injected, a method of subcutaneously implanting a sustained-release preparation of gelatine into which midkine protein is injected, a method of administering a virus vector bearing midkine genes into the coronary vessel flow, and a method of directly injecting midkine protein using an osmotic pump.

The sustained-release capsules of the fine hydroxyapatite particles as described above can be produced by methods well-known to those skilled in the art. Specifically, the sustained-release capsules can be produced by filling pores in fine hydroxyapatite particles with midkine protein, human serum protein and mucopolysaccharide, and then further adding divalent metallic ions to plug the pores, as described in Japanese Patent Publication No. 2004-75662. The sustained-release preparation of gelatine as described above can be produced by methods well-known to those skilled in the art. Specifically, the sustained-release preparation can be produced by trickle impregnation of an aqueous solution containing midkine protein into a cross-linked gelatin gel made to be water-insoluble by cross-linking treatment of gelatine which is a naturally-occurring polymer which can be decomposed and absorbed in the body, as described in U.S. Pat. No. 3,639,593. The sustained-release preparation may be also produced by suspending the cross-linked gelatin gel into the aqueous solution containing the midkine protein to swell again.

Pharmaceutically acceptable additives such as preservatives and stabilizers as described above may be also added to the sustained-release capsules of the present invention.

Virus vectors as described above include virus vectors from recombinant adenoviruses, retroviruses, and the like. More specifically, a midkine gene can be introduced into cells by the steps of: introducing the MK gene into DNA or RNA viruses such as detoxicated retroviruses, adenoviruses, adeno-associated viruses, herpesviruses, vaccinia viruses, poxviruses, polioviruses, sindbis viruses, Sendai viruses, SV40 or immunodeficiency viruses (HIV); and then infecting the cells with the resultant recombinant virus. In the present invention, methods for introducing an agent into a subject through virus vectors include: in vivo introduction method comprising directly introducing an agent into the body through virus vectors; and ex vivo introduction method comprising removing a cell out of a subject to introduce an agent into the cell through virus vectors outside of the body, and then reintroducing the cell into the body.

All prior art documents cited herein are hereby incorporated by reference.

### EXAMPLES

The present invention is more specifically described below as Examples, but the present invention is not limited thereto.

### Example 1

### Angiogenic Activity of Midkine

First, it was examined whether or not a growth factor midkine showed angiogenic activity.

Specifically, angiogenic activities were compared through subcutaneous matrigel injection of midkine into mice. The subcutaneous matrigel injection of midkine with a concentration of 500 ng/ml or angiogenesis factor bFGF (positive control) with a concentration of 500 ng/ml was carried out into mice to observe the angiogenic activity. HE (hematoxylin eosin) staining of the section of subcutaneous matrigel was carried out to measure the number of blood vessels.

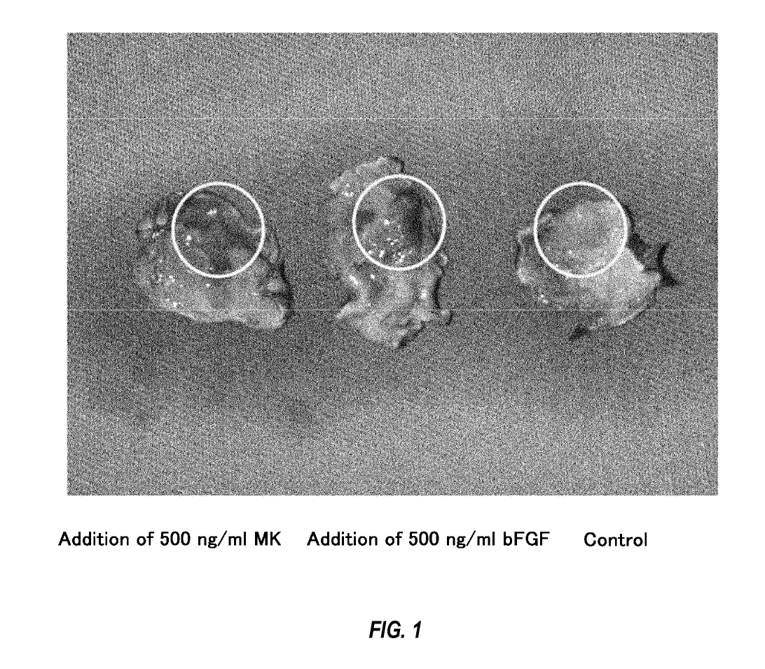

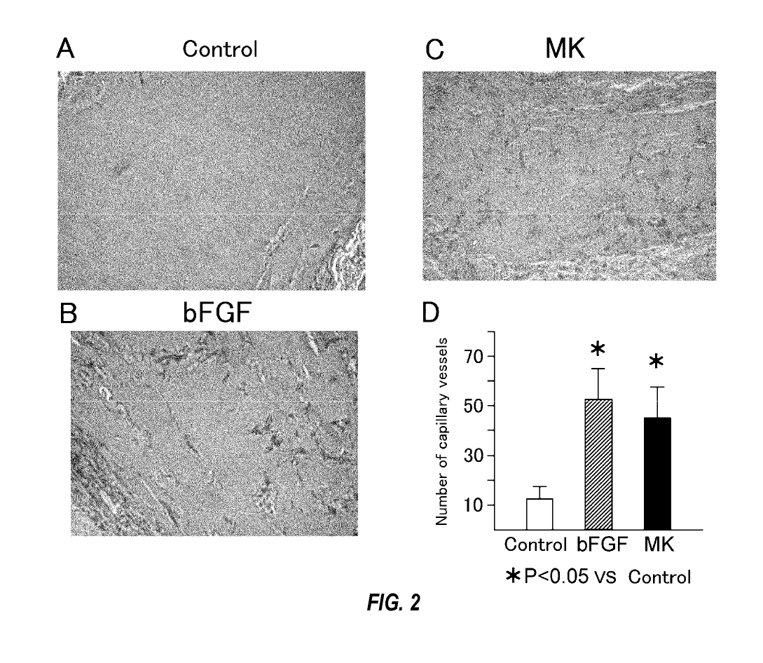

As a result, the remarkable proliferation of the blood vessels by the addition of the midkine was observed in comparison with controls (FIG. 1). In addition, the number of blood vessels measured at 7 days after the administration exhibited a significant increase in the number of blood vessels compared to the controls receiving nothing (FIG. 2; 3.31±0.18 fold vs. controls, n=7, p<0.05) . All the mice receiving midkine showed the results similar to those of the mice receiving bFGF used as the positive control.

### Example 2

### Lumen Formation Activity of Midkine Subsequently, it was examined whether or not midkine had a lumen formation activity in cultured vascular endothelial cells.

Midkine at a concentration of 100 ng/ml was added to HUVECs (normal human umbilical vein endothelial cells), cultured on growth factor-deficient matrigel in the absence of blood serum for 6 hours, and its influence on lumen formation was observed.

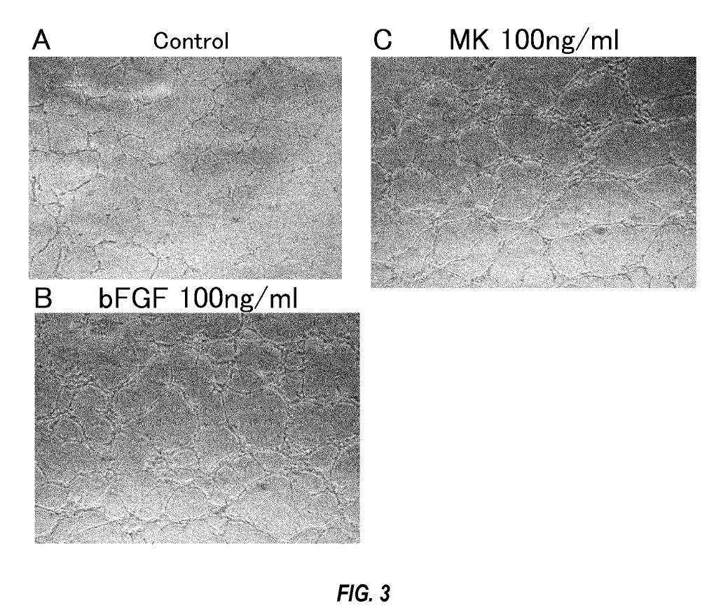

As a result, the lumen formation was found to be promoted by the midkine, similarly as in case of the positive control bFGF (FIG. 3). These results have strongly corroborated that midkine has angiogenic activity.

### Example 3

### Examination of Therapeutic Effect of Midkine in Lower Extremity Ischemic Disease Model Mice

Subsequently, the therapeutic effect of midkine was examined in lower extremity ischemic disease model mice. Specifically, the lower extremity ischemic disease model mice were produced, and the blood flow of the lower extremity was measured in each of the midkine-knockout and wild-type mice.

The midkine-knockout mice (Mdk^{-/-} mice) that were bred by a method described in the Nakamura et al. (Genes Cells 3:811-822, 1998) were used. Both of the wild-type (Mdk^{+/+} mice) and midkine-knockout (Mdk^{-/-} mice) mice had a genetic background of C57BL/6. In addition, the mice of the same age were used in the study.

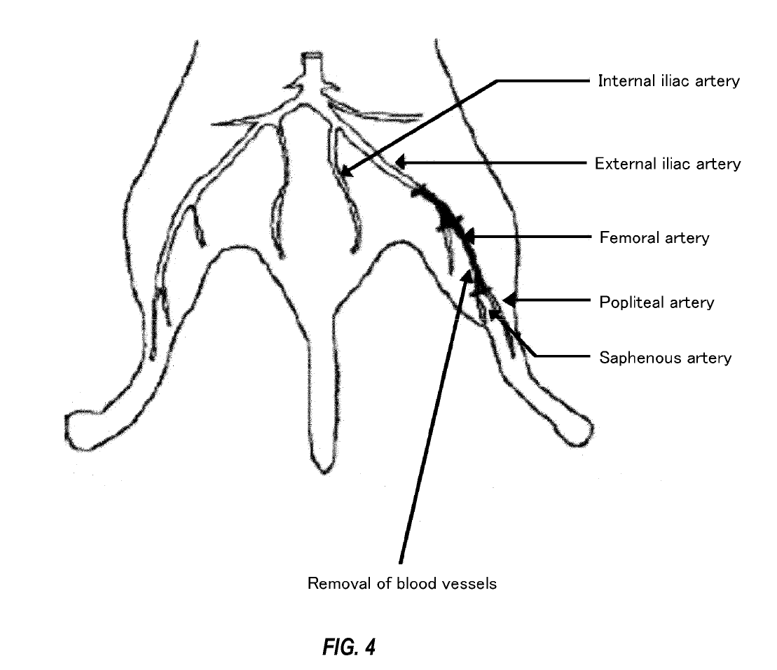

The lower extremity ischemic disease model mice were produced by removing the blood vessels from the femoral artery through the external iliac artery of each of the midkine-knockout mice (MKKO) and the wild-type mice (WT) as shown in FIG. 4.

The blood flow of the lower extremity of each mouse was measured by a laser Doppler blood flowmeter on postoperative day 14 of removing the blood vessels to calculate the rate (L/R rate) of ischemic posterior limb blood flow/non-ischemic posterior limb blood flow.

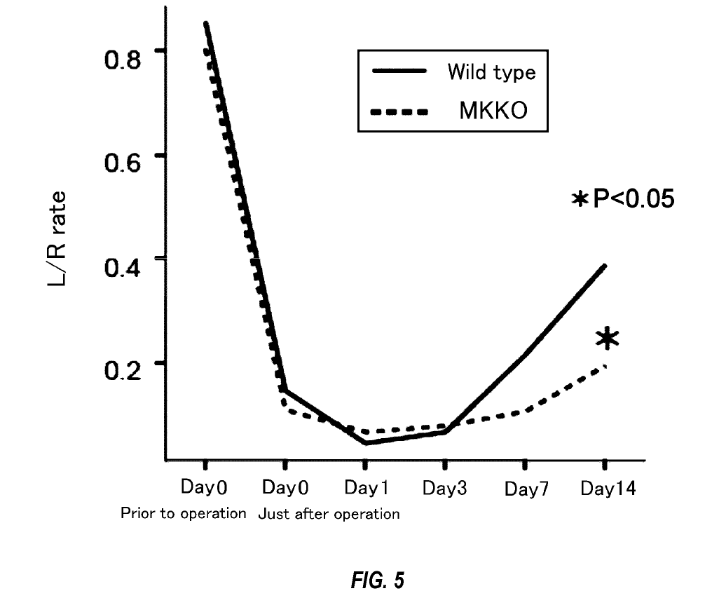

As a result, on the postoperative day 14, the blood flows of the midkine-knockout mice were found to significantly decrease compared to the wild-type mice (FIG. 5; 0.19±0.01in MKKO vs. 0.41±0.03 in WT, n=9, p<0.05).

This result revealed that midkine is involved in peripheral revascularization.

### Example 4

### Introduction of Midkine into Ischemic Lower Extremity

Subsequently, adenovirus vectors were used to introduce midkine into the ischemic lower extremity muscles of the lower extremity ischemic disease model wild-type mice. The operation of removal of the blood vessels was carried out, followed by injecting 10 microl of adenovirus vectors including DNA encoding midkine, at a concentration of 5×10⁹ pfu/ml, into ten sites of the adductor muscle of the thigh per mouse to measure the blood flow of the lower extremity by the same method as in Example 3.

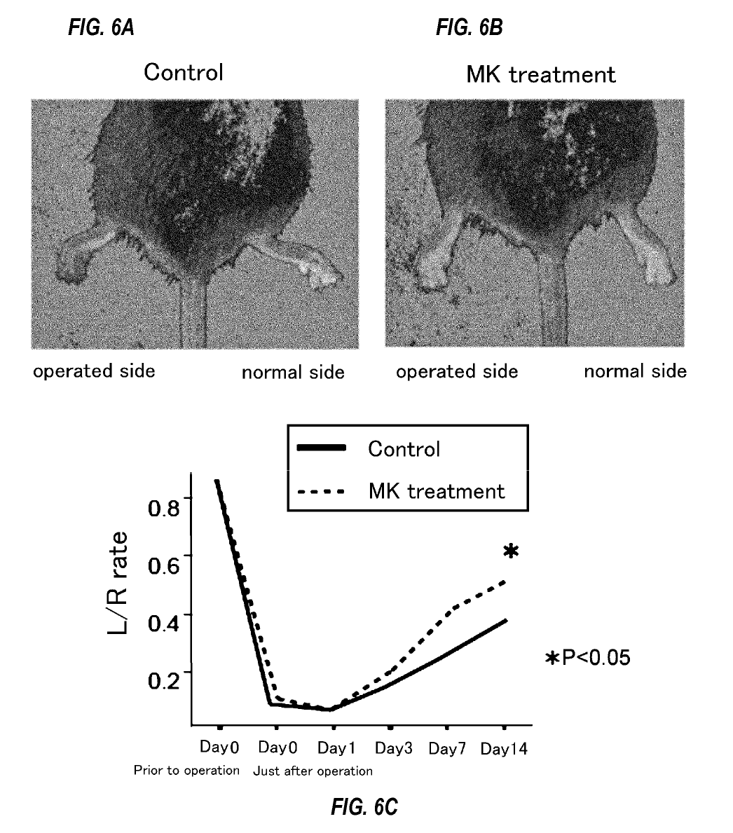

As a result, significant improvement in blood flow was observed in the group into which the midkine was introduced, compared to the non-treatment group (group receiving an injection of only adenovirus vectors) (FIG. 6).

### Example 5

### Introduction of Midkine Through Sustained-Release

### Preparation Using Fine Hydroxyapatite Particles or Gelatine

Midkine was introduced into the ischemic lower extremity muscle of the lower extremity ischemic model rat through a sustained-release preparation using fine hydroxyapatite particles or gelatine. The operation of removal of the blood vessels was carried out, followed by injecting 0.5 g of heparin apatite containing midkine (MK-Hap, midkine content of 2%) into ten sites in the adductor muscle of the thigh of the rat ischemic lower extremity. Only 0.5 g of heparin apatite was injected into the controls in the same manner.

In addition, 2 mg of gelatine and 10 microg of MK were mixed, followed by incubating the mixture at 37° C. for 2 hours and then the incubated mixture was injected into ten sites in the adductor muscle of the thigh of the rat ischemic lower extremity. Only 2 mg of gelatine was injected into the controls in the same manner after the incubation. The blood flows of the lower extremities were measured by the same method as in Example 3.

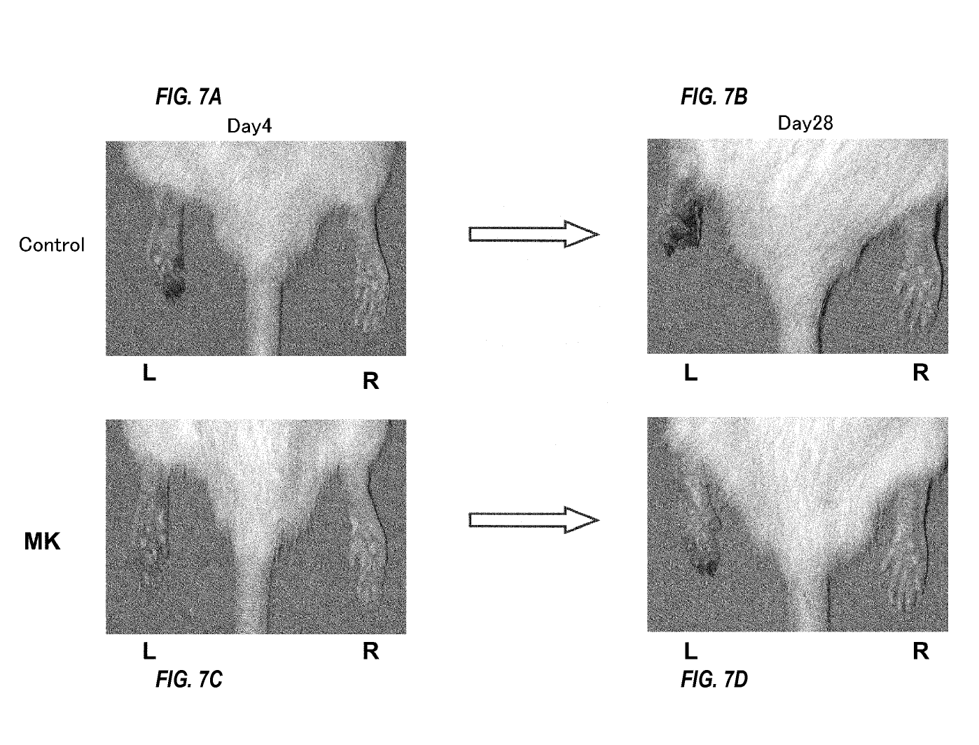

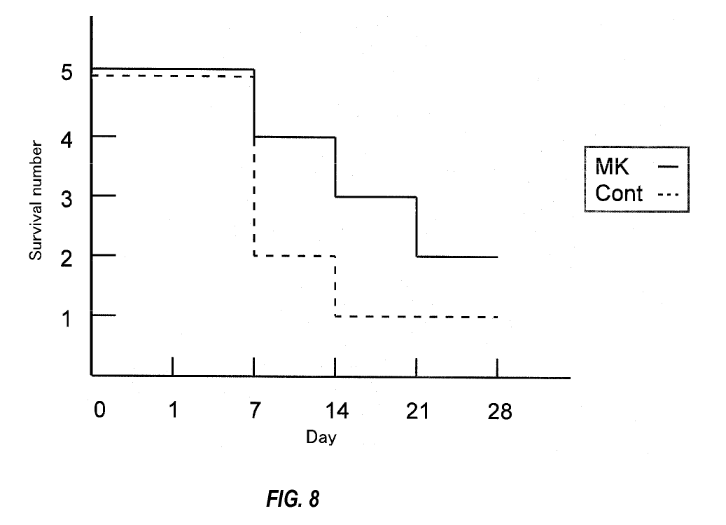

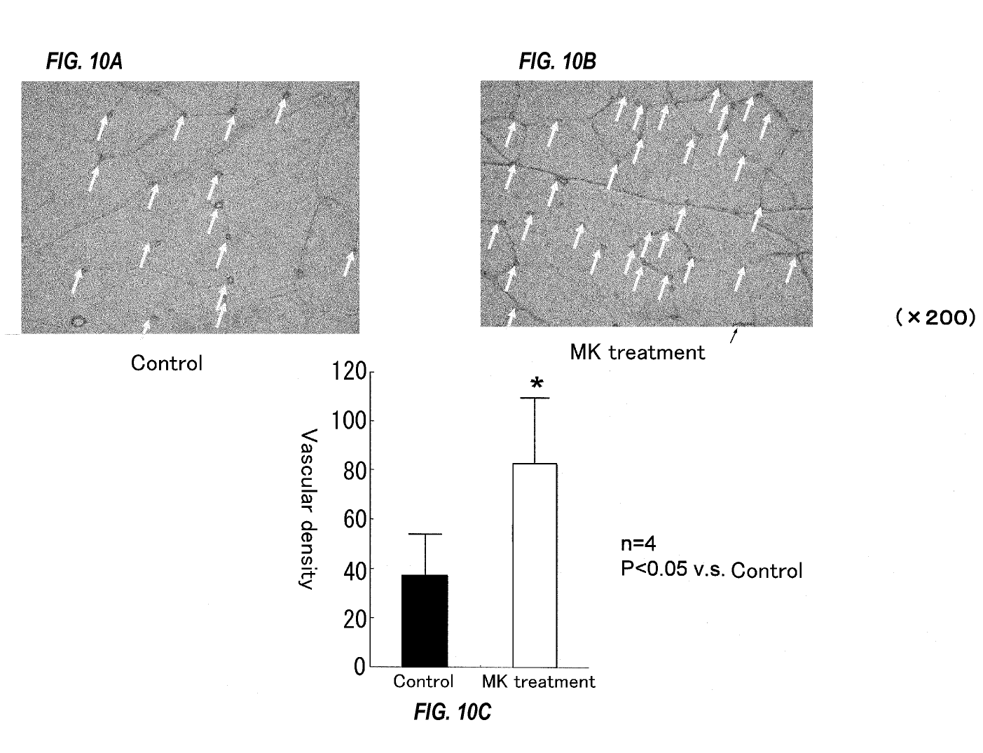

As a result, the midkine protein was found to promote angiogenesis and to permit prevention of the necrosis of the lower extremities by mixing the midkine protein with heparin apatite and gelatine to slowly release the mixture (FIGS. 7, 8 and 10).

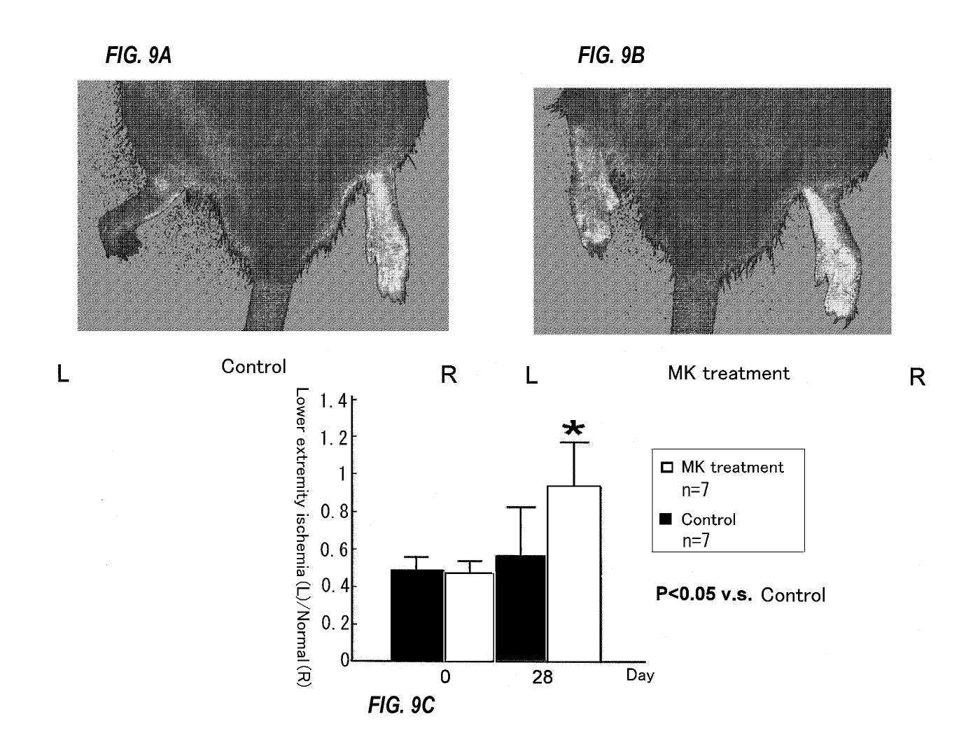

In addition, the use of any sustained-release preparation in the rat lower extremity ischemic model was found to exhibit the notable blood flow improvement effect by midkine protein at 40 microg/kg (FIG. 9).

### Example 6

### Morphologic Evaluation of Angiogenesis Due to Midkine

Subsequently, the morphologic evaluation of the angiogenesis due to the midkine was carried out. The 3-day continuous subcutaneous injections of midkine protein (MK treatment group) and VEGF protein (VEGF treatment group) into the left ear of mice were carried out at 10 microg/ml and 20 microl, respectively. The 3-day continuous subcutaneous injection of 20 microl of physiological saline solution into the right ear of the mice was carried out as controls.

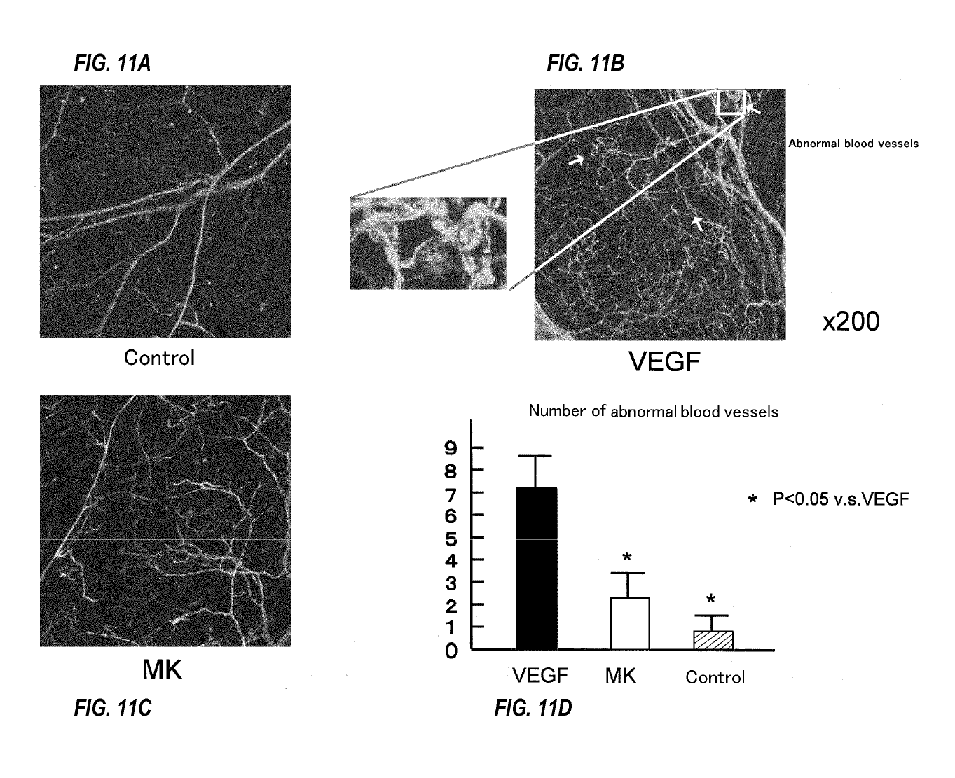

As a result, the continuous administration of the midkine to the auricles of the mice was found to increase the number of microvessels (FIG. 11). In addition, the malformation (vascular malformation) of the microvessels generated by the midkine was small compared to the continuous VEGF treatment group (FIG. 11).

### INDUSTRIAL APPLICABILITY

In the present invention, administration of midkine was found to promote angiogenesis in lesions of occlusive peripheral vascular diseases to improve the symptoms of ischemic limb diseases.

Application of the angiogenic action of midkine to therapies achieves prevention of disease states before reaching the stage of amputation of the upper and lower extremities even in patients with severe limb ischemia, which is resistant to conventional therapies or for which the therapies are unapproved. Thereby, the quality of life of patients with chronic limb ischemia can be expected to be notably improved to consequently improve the vital prognosis for the patients.

[Sequence Listing]

1-4. (canceled)

5. A method of treating or preventing an occlusive peripheral vascular disease, comprising a step of administering midkine or an analog thereof to a subject in need of such treatment or prevention.

6. The method of claim 5, within the midkine or analog is administered as a sustained-release preparation with fine hydroxyapatite particles or gelatine.

7. The method of claim 5, wherein the midkine or analog is administered by intravenous or intramuscular injection.

8. A method of treating or preventing an occlusive peripheral vascular disease, comprising a step of administering a virus vector comprising at least one DNA molecule that encodes midkine or an analog thereof.

9. The method of claim 5, wherein the occlusive peripheral vascular disease is based on arteriosclerosis obliterans, Buerger's disease or diabetic angiopathy.

10. The method of claim 6, wherein the midkine or analog is administered by intravenous or intramuscular injection.

11. The method of claim 6, wherein the occlusive peripheral vascular disease is arteriosclerosis obliterans, Buerger's disease or diabetic angiopathy.

12. The method of claim 7, wherein the occlusive peripheral vascular disease is arteriosclerosis obliterans, Buerger's disease or diabetic angiopathy.

13. The method of claim 8, wherein the occlusive peripheral vascular disease is arteriosclerosis obliterans, Buerger's disease or diabetic angiopathy.

14. A pharmaceutical composition that comprises a sustained-release preparation of midkine or an analog thereof with fine hydroxyapatite particles or gelatine.

15. A pharmaceutical composition that comprises a virus vector comprising at least one DNA molecule that encodes midkine or an analog thereof.
